(19) United States
(12) Patent Application Publication
(10) Pub. No.: US 2007/0275923 A1
(43) Pub. Date: Nov. 29, 2007

(54) CATIONIC PEPTIDES FOR siRNA INTRACELLULAR DELIVERY

(75) Inventors: Lishan Chen, Bellevue, WA (US); Michael E. Houston, Sammamish, WA (US); Roger C. Adami, Snohomish, WA (US); James Anthony McSwiggen, Bothell, WA (US); Sasha J. Mayer, Snohomish, WA (US); Steven C. Quay, Woodinville, WA (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/676,221

(22) Filed: Feb. 16, 2007

Related U.S. Application Data
(60) Provisional application No. 60/803,175, filed on May 25, 2006.

(51) Int. Cl. A61K 31/7052; C07H 21/04; C12N 15/00; C12N 5/06

(52) U.S. Cl. 514/44; 435/375; 435/440; 536/23.1

(57) ABSTRACT

What is described is a composition for delivery of a RNA molecule to a cell, comprising: a double stranded RNA (dsRNA) molecule of about 15 to about 40 base pairs; and a polynucleotide delivery-enhancing peptide, comprising a region of alternating lysine and histidine residues, or of alternating D and L forms of arginine.

This patent application claims priority under 35 U.S.C. § 119(e) of U.S. Provisional Application No. 60/803,175 filed May 25, 2006, the contents of which are incorporated herein by reference.

### BACKGROUND OF THE INVENTION

Delivering nucleic acids into animal and plant cells to achieve a specific biological effect has long been an important object of molecular biology research and development. Recent developments in the areas of gene therapy, antisense therapy and RNA interference (RNAi) therapy have created a need to develop more efficient means for introducing nucleic acids into cells.

A variety of methods are available for delivering nucleic acids artificially into cells. These include transfection via calcium phosphate, cationic lipid, and lipsomal delivery. Nucleic acids can also be introduced into cells by electroporation and viral transduction. However, there are several disadvantages to these methods including cytotoxicity, antigenicity, complement activation, solubility, blood compatibility and stability.

Thus, there remains a long-standing need in the art for better tools and methods to deliver nucleic acids, peptides and other pharmacological agents into cells, particularly in view of the fact that existing techniques for delivering cargo into cells are limited by poor efficiency and/or high toxicity of the delivery reagents. Related needs exist for improved methods and formulations to deliver an effective amount, in an active and enduring state, and using non-toxic delivery vehicles, to selected cells, tissues, or compartments to mediate regulation of gene expression in a manner that will alter a phenotype or disease state of the targeted cells.

### DETAILED DESCRIPTION OF INVENTION

The present invention satisfies these needs and fulfills additional objects and advantages by providing novel compositions and methods for the introduction of nucleic acids into cells. In particular, the present invention relates to compositions for the intracellular delivery of a short interfering nucleic acid (siNA), or a precursor thereof, in combination with a cationic peptide.

In an embodiment of the instant invention, the siNA may be a short interfering nucleic acid (siNA), a short interfering RNA (siRNA), a double-stranded RNA (dsRNA), micro-RNA (mRNA), or a short hairpin RNA (shRNA).

In an embodiment of the invention, the cationic peptide may be a peptide with a plurality of positively charged residues sufficient to bind with a nucleic acid. In one embodiment of the invention, the cationic peptide is a copolymer comprised of about or around 5 to 20 repeats. Each repeat, for example, is comprised of two or more positively charged residues where at least two different positively charged residues are represented within the repeat. In another embodiment, the cationic peptide is a cationic polyamino acid comprising a plurality of positively charged residues consisting, for example, of alternating D- and L-forms of the positively charged residue.

Further provided are peptides comprising sufficient positively-charged residues to bind to a nucleic acid. The peptides may comprise between about 2 and about 45 positively-charged residues; between about 3 and about 45 positively-charged residues; between about 4 and about 45 positively-charged residues; between about 5 and about 45 positively-charged residues; between about 10 and about 45 positively-charged residues; between about 15 and about 45 positively-charged residues; between about 20 and about 45 positively-charged residues; between about 25 and about 45 positively-charged residues; between about 30 and about 45 positively-charged residues; between about 35 and about 45 positively-charged residues; between about 40 and about 45 positively-charged residues.

In additional embodiments, the peptide may be pegylated to improve stability and/or efficacy, particularly in the context of in vivo administration. In yet another embodiment, the peptide may be acetylated. D-amino acid residues may be employed if desired.

The peptides may be dispersed within a pharmaceutically acceptable medium, bound to a nucleic acid, associated with a matrix, associated with a carrier or a targeting ligand, covalently linked to a targeting ligand, covalently linked to at least a first glycosyl unit, thereby forming a glycopeptide targeting ligand, covalently linked to at least a first oligosaccharide unit to form a glycopeptide targeting ligand, or may be both bound to a nucleic acid and associated with a matrix, carrier or a targeting ligand.

The peptides may thus be summarized as being between about 3 and about 50 amino acids in length, comprising sufficient positively-charged residues to bind to a nucleic acid.

Targeted siRNA delivery complexes comprise a targeting ligand and a nucleic acid complex of nucleic acid and cationic peptides.

Multimolecular complexes of the present invention comprise a carrier, a targeting ligand and a nucleic acid complex of nucleic acid and cationic peptides. The multimolecular complexes may further comprise a biocompatible matrix.

Nucleic acid complexes with a particle size of between about 10 nm and about 100 nm in diameter; between about 10 nm and about 50 nm in diameter; and between about 10 nm and about 20 nm in diameter are included, but are not limiting of the invention.

In another embodiment of the invention, the population of peptides is effective to form a nucleic acid-peptide complex that is substantially stable in an extracellular biological environment and that releases nucleic acids intracellularly in a manner effective to reduce gene expression of a target gene. The cell may be located within an animal, wherein the nucleic acid condensate is administered to the animal.

Methods of preparing a nucleic acid-peptide complex comprise contacting a nucleic acid with cationic peptides that have sufficient positive charge to bind to a nucleic acid. Within the novel compositions of the invention, the siNA may be admixed or complexed with, or conjugated to, the cationic peptide to form a composition that enhances intracellular delivery of the siNA as compared to delivery resulting from contacting the target cells with a naked siNA (i.e., siNA without the delivery-enhancing polypeptide present).

Methods for providing a nucleic acid to an animal comprise providing to the animal an effective amount of a nucleic acid complex that comprises the nucleic acid in functional association with a population of cationic peptides.

Uses of the compositions in accordance with the present invention in the manufacture of medicaments for use to treat a wide range of diseases and disorders amenable to treatment by modification of endogenous gene expression are further encompassed.

### Definitions

As used herein, the term “positively charged amino acid” refers to an amino acid having a positive charge at about pH 3 to about pH 6.

As used herein, the term “positively charged residue” refers to a residue having a positive charge at about pH 3 to about pH 6.

As used herein, the term “cationic peptide” refers to a peptide having a positive charge at about pH 3 to about pH 6.

As used herein, the term “polynucleotide delivery-enhancing peptide” is synomous with “cationic peptide.”

As used herein, the term “copolymer” refers to a polymer peptide comprising 5 to 20 repeats wherein each repeat is comprised of two or more positively charged residues and at least two different positively charged residues are represented within the repeat.

As used herein, the term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule”, refers to any nucleic acid molecule capable of inhibiting or down regulating gene expression or viral replication, for example by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof.

“siNA” means a small interfering nucleic acid, for example a siRNA, that is a short-length double-stranded nucleic acid (or optionally a longer precursor thereof), and which is not unacceptably toxic in target cells. The length of useful siNAs within the invention will in certain embodiments be optimized at a length of approximately 21 to 23 base pairs (bp) long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or following the time of delivery to yield a siNA that is active within the cell to mediate gene silencing. Thus, in certain embodiments, useful siNAs within the invention will have a precursor length, for example, of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 bp in length.

The term “gene” refers to a DNA sequence that comprises control and coding sequences necessary for the production of a peptide or precursor thereof. The peptide can be encoded by a full length coding sequence or by any portion of the coding sequence so long as the desired activity is retained.

As used herein the term “cell” is meant to include both prokaryotic (e.g., bacterial) and eukaryotic (e.g., mammalian or plant) cells. Cells may be of somatic or germ line origin, may be totipotent or pluripotent, and may be dividing or non-dividing. Cells can also be derived from or can comprise a gamete or an embryo, a stem cell, or a fully differentiated cell. Thus, the term “cell” is meant to retain its usual biological meaning and can be present in any organism such as, for example, a bird, a plant, and a mammal, including, for example, a human, a cow, a sheep, an ape, a monkey, a pig, a dog, and a cat. Within certain aspects, the term “cell” refers specifically to mammalian cells, such as human cells, that contain one or more siRNA molecule(s) of the present invention.

As used herein, the term “RNA” is meant to include polynucleotide molecules comprising at least one ribonucleotide residue. The term “ribonucleotide” is meant to include nucleotides with a hydroxyl group at the 2′ position of a β-D-ribo-furanose moiety. The term RNA includes, for example, double-stranded RNAs; single-stranded RNAs; and isolated RNAs such as partially purified RNA, essentially pure RNA, synthetic RNA, recombinantly produced RNA, as well as altered RNA that differ from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. As disclosed in detail herein, nucleotides in the siRNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. These antisense and sense RNAs have been conventionally synthesized with an RNA synthesizer.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. Optionally, the siRNA include single strands or double strands of siRNA.

A siHybrid molecule is a double-stranded nucleic acid that has a similar function to siRNA. Instead of a double-stranded RNA molecule, an siHybrid is comprised of an RNA strand and a DNA strand. Preferably, the RNA strand is the antisense strand as that is the strand that binds to the target mRNA. The siHybrid created by the hybridization of the DNA and RNA strands have a hybridized complementary portion and preferably at least one 3′ overhanging end.

By “highly conserved sequence region” is meant, a nucleotide sequence of one or more regions in a target gene does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly(DL-lactide-coglycolide) microspheres for sustained release delivery after intracerebral implantation (Emerich, D. F., et al., Cell Transplant 8:47-58, 1999, Alkermes, Inc., Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry 23:941-949, 1999). Other non-limiting examples of delivery strategies for the nucleic acid molecules of the instant invention include material described in Boado, et al., J. Pharm. Sci. 87:1308-1315, 1998; Tyler, et al., FEBS Lett. 421:280-284, 1999; Pardridge, et al., PNAS USA 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA 96:7053-7058, 1999.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

A “non-nucleotide” means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymidine, for example at the C1 position of the sugar.

The term “ligand” refers to any compound or molecule, such as a drug, peptide, hormone neurotransmitter that is capable of interacting with another compound, such as a receptor, either directly or indirectly. The receptor that interacts with a ligand can be present on the surface of a cell or can alternately be an intracellular receptor. Interaction of the ligand with the receptor can result in a biochemical reaction, or can simply be a physical interaction or association.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of the invention can comprise an antisense region having length sufficient to mediate RNAi in a T-cell (e.g., about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a T-cell (e.g., about 19 to about 22 (e.g., about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA). For example, the term “modulate” can mean “inhibit,” but the use of the word “modulate” is not limited to this definition.

By “inhibit”, “down-regulate”, “knockdown” or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of the invention. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. In another embodiment, inhibition, down-regulation, or reduction of gene expression with a nucleic acid molecule of the instant invention is greater in the presence of the nucleic acid molecule than in its absence.

Gene “silencing” refers to partial or complete loss-of-function through targeted inhibition of gene expression in a cell and may also be referred to as “knockdown.” Depending on the circumstances and the biological problem to be addressed, it may be preferable to partially reduce gene expression. Alternatively, it might be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of the invention, including prophylactic and therapeutic methods, which will be capable of knocking down target gene expression, in terms of mRNA levels or protein levels or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of a siNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “target nucleic acid” or “nucleic acid target” or “target RNA” or “RNA target” or “target DNA” or “DNA target” is meant any nucleic acid sequence whose expression or activity is to be modulated. The target nucleic acid can be DNA or RNA and is not limited single strand forms.

The term “biologically active molecule” as used herein, refers to compounds or molecules that are capable of eliciting or modifying a biological response in a system. Non-limiting examples of biologically active siNA molecules either alone or in combination with other molecules contemplated by the instant invention include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

By “cap structure” is meant chemical modifications, which have been incorporated at either terminus of the oligonucleotide (see, for example, Adamic, et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Lyer, Tetrahedron 49:1925, 1993; incorporated by reference herein).

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

By “biological system” is meant, material, in a purified or unpurified form, from biological sources, including but not limited to human, animal, plant, insect, bacterial, viral or other sources, wherein the system comprises the components required for RNAi activity. The term “biological system” includes, for example, a cell, tissue, or organism, or extract thereof. The term biological system also includes reconstituted RNAi systems that can be used in an in vitro setting.

The term “biodegradable linker” as used herein, refers to a nucleic acid or non-nucleic acid linker molecule that is designed as a biodegradable linker to connect one molecule to another molecule, for example, a biologically active molecule to a siNA molecule of the invention or the sense and antisense strands of a siNA molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, or uracil joined to the 1′ carbon of beta.-D-ribo-furanose.

By “modified nucleoside” is meant any nucleotide base which contains a modification in the chemical structure of an unmodified nucleotide base, sugar and/or phosphate. Non-limiting examples of modified nucleotides are shown by Formulae I-VII and/or other modifications described herein.

In connection with 2′-modified nucleotides as described for the present invention, by “amino” is meant 2′-NH₂ or 2′-O—NH₂, which can be modified or unmodified. Such modified groups are described, for example, in Eckstein, et al., U.S. Pat. No. 5,672,695 and Matulic-Adamic, et al., U.S. Pat. No. 6,248,878.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Large double-stranded RNA” refers to any double-stranded RNA having a size greater than about 40 bp for example, larger than 100 bp or more particularly larger than 300 bp. The sequence of a large dsRNA may represent a segment of an mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

“One or more dsRNAs” refers to dsRNAs that differ from each other on the basis of sequence.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner, et al., CSH Symp. Quant. Biol. LII, 1987, pp. 123-133; Frier, et al., Proc. Nat. Acad. Sci. USA 83:9373-9377, 1986; Turner, et al., J. Am. Chem. Soc. 109:3783-3785, 1987). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

By “subject” is meant an organism, tissue, or cell, which may include an organism as the subject or as a donor or recipient of explanted cells or the cells that are themselves subjects for siNA delivery. “Subject” therefore may refers to an organism, organ, tissue, or cell, including in vitro or ex vivo organ, tissue or cellular subjects, to which the nucleic acid molecules of the invention can be administered and enhanced by peptides described herein. Exemplary subjects include mammalian individuals or cells, for example human patients or cells.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

In this specification and the appended claims, the singular forms of “a”, “an” and “the” include plural reference unless the context clearly dictates otherwise.

### Small-Interfering Nucleic Acids (siNAs) and the RISC Complex

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2a (eIF2α), while 2′-5′-oligoadenylate synthetase causes nonspecific mRNA degradation via activation of RNase L. By virtue of their small size (referring particularly to non-precursor forms), usually less than 30 base pairs, and most commonly between about 17-19, 19-21, or 21-23 base pairs, the siNAs of the present invention avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place in the middle of the region complementary to the antisense strand of the siRNA duplex. Studies have shown that 21 nucleotide siRNA duplexes are most active when containing two nucleotide 3′-overhangs. Furthermore, complete substitution of one or both siRNA strands with 2′-deoxy(2′-H) or 2′-O-methyl nucleotides abolishes RNAi activity, whereas substitution of the 3′-terminal siRNA overhang nucleotides with deoxy nucleotides (2′-H) has been reported to be tolerated.

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long. Within exemplary embodiments, double-stranded portions of siNAs, in which two strands pair up, are not limited to completely paired nucleotide segments, and may contain nonpairing portions due to mismatch (the corresponding nucleotides are not complementary), bulge (lacking in the corresponding complementary nucleotide on one strand), overhang, and the like. Nonpairing portions can be contained to the extent that they do not interfere with siNA formation. In more detailed embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other cause are also contemplated. Furthermore, in the present invention the double-stranded region of siNAs in which two strands pair up may contain both bulge and mismatched portions in the approximate numerical ranges specified.

The terminal structure of siNAs of the invention may be either blunt or cohesive (overhanging) as long as the siNA retains its activity to silence expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides. The total length of siNAs having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. Furthermore, since the overhanging sequence may have low specificity to a target gene, it is not necessarily complementary (antisense) or identical (sense) to the target gene sequence. Furthermore, as long as the siNA is able to maintain its gene silencing effect on the target gene, it may contain low molecular weight structure (for example a natural RNA molecule such as tRNA, rRNA or viral RNA, or an artificial RNA molecule), for example, in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, often 15 to 35 bp, and more commonly about 21 to 30 bp long. Alternatively, the length of the double-stranded region that is a final transcription product of siNAs to be expressed in a target cell may be, for example, approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example, Martinez, et al., Cell 110:563-574, 2002, and Schwarz, et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In certain embodiments, the short interfering nucleic acid molecules of the invention lack 2′-hydroxy(2′-OH) containing nucleotides. In certain embodiments short interfering nucleic acids do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules for use within the invention may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic interactions, and/or stacking interactions.

siNAs for use within the invention can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

Within additional embodiments, siNAs for intracellular delivery according to the methods and compositions of the invention can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

The siNA molecules described herein, the antisense region of a siNA molecule of the invention can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of a siNA molecule of the invention can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, the invention features a chemically-modified short interfering nucleic acid (siNA) having about 1, 2, 3, 4, 5, 6, 7, 8 or more phosphorothioate internucleotide linkages in one siNA strand. In yet another embodiment, the invention features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the invention can comprise one or more phosphorothioate internucleotide linkages at the 3′-end, the 5′-end, or both of the 3′- and 5′-ends of the sense strand, the antisense strand, or both strands. For example, an exemplary siNA molecule of the invention can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of the invention can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of the invention can comprise one or more (e.g., about 1, 2, 3, 4, 5, 6, 7, 8, 9, 10, or more) purine phosphorothioate internucleotide linkages in the sense strand, the antisense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the antisense strand of the siNA molecules, but also optionally in the sense and/or both antisense and sense strands, comprise modified nucleotides having properties or characteristics similar to naturally occurring ribonucleotides. For example, the invention features siNA molecules including modified nucleotides having a Northern conformation (e.g., Northern pseudorotation cycle, see for example, Saenger, Principles of Nucleic Acid Structure, Springer-Verlag ed., 1984). As such, chemically modified nucleotides present in the siNA molecules of the invention, preferably in the antisense strand of the siNA molecules of the invention, but also optionally in the sense and/or both antisense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, ribothymidine nucleotides and 2′-O-methyl nucleotides.

The sense strand of a double stranded siNA molecule may have a terminal cap moiety such as an inverted deoxybasic moiety, at the 3′-end, 5′-end, or both 3′ and 5′-ends of the sense strand.

Non-limiting examples of conjugates include conjugates and ligands described in Vargeese, et al., U.S. application Ser. No. 10/427,160, filed Apr. 30, 2003, incorporated by reference herein in its entirety, including the drawings. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached both the 3′-end and 5′-end of either the sense strand, the antisense strand, or both strands of the chemically-modified siNA molecule, or any combination thereof. In one embodiment, a conjugate molecule of the invention comprises a molecule that facilitates delivery of a chemically-modified siNA molecule into a biological system, such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese, et al., U.S. Patent Application Publication No. 20030130186, published Jul. 10, 2003, and U.S. Patent Application Publication No. 20040110296, published Jun. 10, 2004. The type of conjugates used and the extent of conjugation of siNA molecules of the invention can be evaluated for improved pharmacokinetic profiles, bioavailability, and/or stability of siNA constructs while at the same time maintaining the ability of the siNA to mediate RNAi activity. As such, one skilled in the art can screen siNA constructs that are modified with various conjugates to determine whether the siNA conjugate complex possesses improved properties while maintaining the ability to mediate RNAi, for example in animal models as are generally known in the art.

An siNA further may be further comprised of a nucleotide, non-nucleotide, or mixed nucleotide/non-nucleotide linker that joins the sense region of the siNA to the antisense region of the siNA. In one embodiment, a nucleotide linker can be a linker of >2 nucleotides in length, for example about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. (See, for example, Gold, et al, Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.)

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma, et al., Nucleic Acids Res. 21:2585, 1993, and Biochemistry 32:1751, 1993; Durand, et al., Nucleic Acids Res. 18:6353, 1990; McCurdy, et al., Nucleosides & Nucleotides 10:287, 1991; Jschke, et al., Tetrahedron Lett. 34:301, 1993; Ono, et al., Biochemistry 30:9914, 1991; Arnold, et al., International Publication No. WO 89/02439; Usman, et al., International Publication No. WO 95/06731; Dudycz, et al., International Publication No. WO 95/11910, and Ferentz and Verdine, J. Am. Chem. Soc. 113:4000, 1991.

The synthesis of a siNA molecule of the invention, which can be chemically-modified, comprises: (a) synthesis of two complementary strands of the siNA molecule; (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol. Bio. 74:59, 1997; Brennan, et al., Biotechnol Bioeng. 61:33-45, 1998; and Brennan, U.S. Pat. No. 6,001,311. Synthesis of RNA, including certain siNA molecules of the invention, follows general procedures as described, for example, in Usman, et al., J. Am. Chem. Soc. 109:7845, 1987; Scaringe, et al., Nucleic Acids Res. 18:5433, 1990; and Wincott, et al., Nucleic Acids Res. 23:2677-2684, 1995; Wincott, et al., Methods Mol. Bio. 74:59, 1997.

The siNAs can be modified extensively to enhance stability by modification with nuclease resistant groups, for example, 2′-amino, 2′-C-allyl, 2′-fluoro, 2′-O-methyl, 2′-H. (For a review see Usman and Cedergren, TIBS 17:34, 1992; Usman, et al., Nucleic Acids Symp. Ser. 31:163, 1994.) SiNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., Eckstein, et al., International Publication No. WO 92/07065; Perrault, et al., Nature 344:565, 1990; Pieken, et al., Science 253:314, 1991; Usman and Cedergren, Trends in Biochem. Sci. 17:334, 1992; Usman, et al., International Publication No. WO 93/15187; and Rossi, et al., International Publication No. WO 91/03162; Sproat, U.S. Pat. No. 5,334,711; Gold, et al., U.S. Pat. No. 6,300,074. All of the above references describe various chemical modifications that can be made to the base, phosphate and/or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications. For a review see Usman and Cedergren, TIBS 17:34, 1992; Usman, et al., Nucleic Acids Symp. Ser. 31:163, 1994; Burgin, et al., Biochemistry 35:14090, 1996. Sugar modification of nucleic acid molecules have been extensively described in the art. See Eckstein, et al., International Publication PCT No. WO 92/07065; Perrault, et al., Nature 344:565-568, 1990; Pieken, et al., Science 253:314-317, 1991; Usman and Cedergren, Trends in Biochem. Sci. 17:334-339, 1992; Usman, et al., International Publication PCT No. WO 93/15187; Sproat, U.S. Pat. No. 5,334,711; and Beigelman, et al., J. Biol. Chem., 270:25702, 1995; Beigelman, et al., International PCT publication No. WO 97/26270; Beigelman, et al., U.S. Pat. No. 5,716,824; Usman, et al., U.S. Pat. No. 5,627,053; Woolf, et al., International PCT Publication No. WO 98/13526; Thompson, et al., Karpeisky, et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina, et al., Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications can be used as described herein to modify the siNA nucleic acid molecules of the instant invention so long as the ability of siNA to promote RNAi in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, “Nucleic Acid Analogues: Synthesis and Properties,” Modern Synthetic Methods, VCH, 1995, pp. 331-417, and Mesmaeker, et al., “Novel Backbone Replacements for Oligonucleotides,” Carbohydrate Modifications in Antisense Research, ACS, 1994, pp. 24-39.

### Supplemental or Complementary Methods of Delivery

Supplemental or complementary methods for delivery of nucleic acid molecules for use within then invention are described, for example, in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any nucleic acid molecule contemplated within the invention.

Nucleic acid molecules and peptides can be administered to cells by a variety of methods known to those of skill in the art, including, but not restricted to, administration within formulations that comprise the siNA and peptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the peptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262.

The compositions of the instant invention can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

Thus within additional embodiments the invention provides pharmaceutical compositions and methods featuring the presence or administration of one or more polynucleic acid(s), typically one or more siNAs, combined, complexed, or conjugated with a peptide, optionally formulated with a pharmaceutically-acceptable carrier, such as a diluent, stabilizer, buffer, and the like.

The present invention satisfies additional objects and advantages by providing short interfering nucleic acid (siNA) molecules that modulate expression of genes associated with a particular disease state or other adverse condition in a subject. Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of the invention may be targeted to lower expression of one gene, which can result in upregulation of a “downstream” gene whose expression is negatively regulated by a product or activity of the target gene.

This siNAs of the present invention may be administered in any form, for example transdermally or by local injection. Comparable methods and compositions are provided that target expression of one or more different genes associated with a selected disease condition in animal subjects, including any of a large number of genes whose expression is known to be aberrantly increased as a causal or contributing factor associated with the selected disease condition.

Negatively charged polynucleotides of the invention (e.g., RNA or DNA) can be administered to a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez, et al., Bioconjugate Chem. 10:1068-1074, 1999; Wang, et al., International PCT Publication Nos. WO 03/47518 and WO 03/46185), poly(lactic-co-glycolic acid) (PLGA) and PLCA microspheres (see for example U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res. 5:2330-2337, 1999, and Barry, et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a subject.

Any one or combination of the cationic peptides of the present invention may be selected or combined to yield effective polynucleotide delivery-enhancing polypeptide reagents to induce or facilitate intracellular delivery of siNAs within the methods and compositions of the invention.

### Pharmaceutical Formulations

The present invention also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

### EXAMPLES

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### Example 1

### Materials and Methods Used

The present example illustrates the materials and methods used to assess the efficacy of cationic peptides of the present invention to mediate intracellular delivery of siRNA in vitro. The cell culture conditions and protocols for each assay are explained below in detail. The protocols described below are for demonstrative purposes only and may be changed and/or modified accordingly.

siRNA Preparation

siRNA was suspended in Hyclone nuclease free water at a concentration of 5 mg/mL based on dry weight. The actual siRNA stock concentration was measured by making a 1 to 2000 and a 1 to 100 dilution of stock in water and measuring A260 on an Eppendorf UV Spectrophotometer. Quantification of concentration was made by calculating a ratio of the absorbance of these dilutions to an absorbance at 260 nm of 1.000 for 38.5 μg/mL of siRNA. This concentrated stock solution was stored in single-use aliquots at −80° C. and diluted to 20 μg/mL (2× concentration) for this assay. The nucleotide sequences of two exemplary siRNAs used in the present invention are G1498 (5′-ggaucuuauuucuucggag-3′; SEQ ID NO: 1) and LC20 (5′-gggucggaacccaagcuua-3′; SEQ ID NO: 2). The sequences shown may be modified accordingly, i.e., 3′ overhangs and/or the introduction of modified nucleosides.

### Cell Cultures

Mouse tail fibroblast (MTF) cells were derived from the tails of C57BL/6J mice. Tails were removed, immersed in 70% ethanol and then cut into small sections with a razor blade. The sections were washed three times with PBS and then incubated in a shaker at 37° C. with 0.5 mg/mL collagenase, 100 units/mL penicillin and 100 μg/mL streptomycin to disrupt tissue. Tail sections were then cultured in complete media (Dulbecco's Modified Essential Medium with 20% FBS, 1 mM sodium pyruvate, nonessential amino acids and 100 units/mL penicillin and 100 μg/mL streptomycin) until cells were established. Cells were cultured at 37° C., 5% CO₂ in complete media as outlined above.

### Transfection

Cells were be plated per well in a microtiter plate the day before transfection in complete media. The siRNA was be labeled, for example with a FAM, Alexa488, Alexa647 or Cy-5 conjugate. The cationic polymer peptide and siRNA were mixed in the microtiter plate complex with cells previously with OptiMEM. 7-AAD was added to each transfection to measure cell viability. Cells were transfected for three hours at 37° C., 5% CO₂. The transfection mixture was removed and the cells were washed with TE buffer for about 10 minutes at 37° C., 5% CO₂. Transfected cells were transferred to a V bottom plate and centrifuged. The TE buffer was removed and the transfected cells were resuspended in fluorescence activated cell sorting (FACS) buffer.

### Gold Dye Displacement Assay

The relative binding of various peptides to siRNA via a rapid screen is assessed by indirect measurement of the displacement of SYBR-gold nucleic acid binding dye. A buffered mixture of siRNA, peptide and SYBR-gold is prepared in the measurement plate in duplicate such that the peptide and SYBR-gold dye undergoes simultaneous competitive binding of the siRNA. The concentration of siRNA is fixed at 10 μg/mL and is combined with a titration of each peptide ranging in a concentration that corresponds to a peptide:siRNA charge ratio between 0.05 to 10. Since SYBR-gold dye only fluoresces when bound to siRNA, peptide binding to the siRNA is inhibits binding of the dye and consequently reduces the fluorescence. Therefore, the amount of fluorescence correlates inversely to the binding of the peptide to the siRNA. Both Kd and Bₘₐₓ values will be calculated. A greater Kd value indicates greater binding affinity between the peptide and the siRNA.

SYBR-gold nucleic acid binding dye stock, a 10,000× concentrate, was supplied by Invitrogen (Carlsbad, Calif.) and stored at −20° C. The concentrate is allowed to equalibrize to room temperature before diluting 1 to 100 in Hyclone nuclease free water. This is diluted 1 to 10 in the experimental plate for a final concentrate of 10× for the assay. This is the optimal dilution to achieve linear binding to siRNA duplex at a concentration range of up to 50 μg/mL concentration. The values used to generate the standard curve demonstrating linear binding of SYBR-gold to G1498 siRNA is shown in Table 1.

TABLE 1
G1498 siRNA Standard Curve Values

| [G1498] | Mean | |
|---|---|---|
| μg/mL | Fluorescence | Std Dev |
| 0 | 0 | 3.86 |
| 1.56 | 376 | 10.0 |
| 3.13 | 840 | 44.8 |
| 6.25 | 3254 | 91.4 |
| 12.5 | 10591 | 762 |
| 25.0 | 26276 | 1497 |
| 50.0 | 36543 | 240 |

Samples are mixed directly in the 384 well analysis plate. First, 5 μL SYBR-gold dye is pipetted into each well with a multichannel pipet, touching the tip to the bottom of the well to draw out the solution completely. Second, 22.5 μL of 2× peptide solution is added with a single channel pipet. Finally, 22.5 μL of 2×siRNA is added with a multichannel pipet. The plate is covered immediately with foil and tapped gently to mix and draw down any droplets on the side of the well.

Fluorescence is measured using the SpectraMax fluorescent plate reader from Molecular Devices, Sunnyvale, Calif. Plate settings include shaking before reading, one read per well, with excitation wavelength of 495 nm and emission wavelength of 537 nm. The plate is read within 30 minutes of the addition of the siRNA.

### Scatchard Plot

Scatchard Plot is a plot of peptide binding ([peptide]bound/[peptide]free) vs. [peptide]bound. The slope of the linear regression of this plot is −1/Kd and Bₘₐₓ is the y-intercept. Since the concentration of free and bound peptide cannot be measured directly, indirect measurements of siRNA is used for the calculation. Free siRNA is determined from measured fluorescence using the standard curve. Bound siRNA is determined from the standard curve by mass balance from the known initial siRNA concentration (10 μg/mL). Bound peptide is calculated from bound siRNA by assuming the (siRNA:Peptide) bound molar ratio is equal to the (siRNA:Peptide) charge ratio for a single molecule pairing. From this calculated bound peptide amount, the free peptide is calculated by mass balance.

### Fluorescence Activated Cell Sorting (FACS):

SiRNA cell uptake was measured by FACS.

### Cationic Peptides:

Table 2 below illustrates the cationic peptides, their respective counterion and molecular weights, screened for siRNA intracellular delivery efficacy and their effect on cell viability in vitro. Within Table 2, the abbreviations “Ac” means the peptide is acetylated, “PEG2K” means the peptide is conjugated with poly(ethylene glycol) (PEG) with a molecular weight of 2 kiloDaltons (kD), “PEG 10K” means the peptide is conjugated with a 10 kD PEG, “Mal” means maleimido and for the peptide PN912 only “Xaa” means D-arginine.

TABLE 2

Cationic Peptides

| | | | |
|---|---|---|---|
| | | | Molecu- |
| Peptide | | Count- | lar |
| Name | Amino Acid Sequence | erion | Weight |

| | | | |
|---|---|---|---|
| PN986 | PEG (10 KDa)-Mal-Trp- | TFA | 13746.85 |
| | (Lys)₃₀-amide | | |
| | (SEQ ID NO: 3) | | |
| PN927 | PEG (10 KDa)-Mal-(Lys)₂₅- | Acetate | 12919.75 |
| | amide | | |
| | (SEQ ID NO: 4) | | |
| PN925 | PEG (2 KDa)-Mal-(Lys)₂₅- | Acetate | 5513.3 |
| | amide | | |
| | (SEQ ID NO: 4) | | |
| PN928 | PEG (10 KDa)-Mal-(Lys)₂₀- | Acetate | 12296.5 |
| | amide | | |
| | (SEQ ID NO: 5) | | |
| PN926 | PEG (2 KDa)-Mal-(Lys)₂₀- | Acetate | 4873.5 |
| | amide | | |
| | (SEQ ID NO: 5) | | |
| PN931 | PEG (10 KDa)-Mal-(Lys)₁₅- | Acetate | 11655.6 |
| | amide | | |
| | (SEQ ID NO: 6) | | |
| PN930 | PEG (2 KDa)-Mal-(Lys)₁₅- | Acetate | 4232.6 |
| | amide | | |
| | (SEQ ID NO: 6) | | |
| PN932 | NH₂-(Lys)₁₀-amide | Acetate | 1299.7 |
| | (SEQ ID NO: 7) | | |
| PN934 | PEG (10 KDa)-Mal-(Lys)₁₀- | Acetate | 11014.7 |
| | amide | | |
| | (SEQ ID NO: 7) | | |
| PN933 | PEG (2 KDa)-Mal-(Lys)₁₀- | Acetate | 3591.7 |
| | amide | | |
| | (SEQ ID NO: 7) | | |
| PN935 | NH₂-(Lys-His)₁₅-amide | TFA | 3997.7 |
| | (SEQ ID NO: 8) | | |
| PN936 | PEG (2 KDa)-Mal-(Lys- | Acetate | 6289.7 |
| | His)₁₅-amide | | |
| | (SEQ ID NO: 8) | | |
| PN938 | NH₂-(Lys-His)₁₀-amide | TFA | 2671.1 |
| | (SEQ ID NO: 9) | | |
| PN939 | PEG (2 KDa)-Mal-(Lys- | Acetate | 4962.1 |
| | His)₁₀-amide | | |
| | (SEQ ID NO: 9) | | |
| PN940 | PEG (10 KDa)-Mal-(Lys- | Acetate | 12385.1 |
| | His)₁₀-amide | | |
| | (SEQ ID NO: 9) | | |
| PN912 | NH2-(Xaa-Arg)₁₀-amide | HCl | 3142.15 |
| | (SEQ ID NO: 10) | | |
| PN937 | PEG (10 KDa)-Mal-(Lys- | Acetate | 13712.7 |
| | His)₁₅-amide | | |
| | (SEQ ID NO: 8) | | |
| PN986 | PEG (10 KDa)-Mal-Trp- | TFA | 13746.85 |
| | (Lys)₃₀-amide | | |
| | (SEQ ID NO: 3) | | |
| PN951 | NH2-(His)₆-Arg-Ser-Val- | Acetate | 3903.5 |
| | Cys-Arg-Gln-Ile-Lys-Ile- | | |
| | Cys-Arg-Arg-Arg-Gly-Gly- | | |
| | Cys-Tyr-Tyr-Lys-Cys-Thr- | | |
| | Xaa-Arg-Pro-Tyr-amide | | |
| | (SEQ ID NO: 11) | | |
| PN969 | NH₂-(Lys)₉-Gly-Leu-Phe- | TFA | 3207.8 |
| | Gly-Ala-Ile-Ala-Gly-Phe- | | |
| | Ile-Glu-Xaa-Gly-Trp-Glu- | | |
| | Gly-Met-Ile-Asp-Gly-amide | | |
| | (SEQ ID NO: 12) | | |
| PN970 | PEG (10 KDa)-Mal-(Lys)₉₋ | TFA | 12922.8 |
| | Gly-Leu-Phe-Gly-Ala-Ile- | | |
| | Ala-Gly-Phe-Ile-Glu-Xaa- | | |
| | Gly-Trp-Glu-Gly-Met-Ile- | | |
| | Asp-Gly-amide | | |
| | (SEQ ID NO: 12) | | |
| PN947 | Mal-(Lys)₈-Ser-Gln-Pro- | TFA | 3485 |
| | Asp-Ile-Met-Gly-Glu-Trp- | | |
| | Gly-Xaa-Glu-Ile-Phe-Gly- | | |
| | Ala-Ile-Ala-Gly-Phe-Leu- | | |
| | Gly-amide | | |
| | (SEQ ID NO: 13) | | |
| PN183 | NH2-Lys-Glu-Thr-Trp-Trp- | TFA | 3781.2 |
| | Glu-Thr-Trp-Trp-Thr-Glu- | | |
| | Trp-Ser-Gln-Pro-Gly-Arg- | | |
| | Lys-Lys-Arg-Arg-Gln-Arg- | | |
| | Arg-Arg-Pro-Pro-Gln-amide | | |
| | (SEQ ID NO: 14) | | |

### Example 2

### Efficacy of siRNA Intracellular Delivery by Cationic Peptides In Vitro

The present example demonstrates that select cationic peptides of the present invention mediate effective intracellular delivery of siRNA without negatively impacting cell viability. For the instant example, cell-uptake, mean fluorescent intensity (MFI) and cell viability was measured for each peptide. siRNA cell-uptake was measured by the presence of intracellular labeled siRNA. The cell-uptake assay determined the percentage of cells that contain the labeled siRNA. Mean Fluorescence Intensity (MFI) was used to determine the relative mean quantity of labeled siRNA that entered the cells.

Each cationic peptide was mixed with 200 nM of siRNA at 1 μM, 10 μM and 100 μM, which results in a peptide:siRNA molar ratio of 5, 50 and 500, respectively. LIPOFECTAMINE™ mediated siRNA transfection served herein as a positive control for cell-uptake, MFI and cell viability. A non-transfection, siRNA alone, control was also performed. The peptide PN183 served herein as a positive cell-uptake control. Only those peptides that exhibited efficient cell-uptake with no significant effect on cell viability were further characterized for siRNA MFI. Thus, not all the peptides listed in Table 3 below have MFI results.

The cell-uptake, cell viability and MFI results for the cationic peptides is shown below in Table 3. Cell-uptake is expressed as a percentage of the total cells with intracellular labeled siRNA. Cell Viability is also expressed as a percentage where 100% represents 100% viability. The MFI value is a relative value with a higher MFI value indicating a greater amount of labeled siRNA entered the cell. Within Table 3, “ND” means no data.

TABLE 3
Summary of Cationic Peptide siRNA Intracellular Delivery Results

| | Peptide | | % Cell | |
|---|---|---|---|---|
| Peptide Name | Concentration | % Cell-Uptake | Viability | MFI |
| siRNA alone | N/A | 0.5 | 98.1 | 1.15 |
| Lipofectamine | 1 μM | 40 | 97.5 | 8.33 |
| | 10 μM | 51 | 97.8 | 15.9 |
| | 100 μM | 66.2 | 98.5 | 22.8 |
| PN986 | 1 μM | 0.6 | 97.4 | ND |
| | 10 μM | 0.8 | 97.2 | ND |
| | 100 μM | 2.5 | 93.6 | ND |
| PN927 | 1 μM | 1.6 | 95.6 | ND |
| | 10 μM | 0.8 | 97 | ND |
| | 100 μM | 0.7 | 97.3 | ND |
| PN925 | 1 μM | 5.2 | 96.6 | ND |
| | 10 μM | 2.9 | 97.8 | ND |
| | 100 μM | 2.2 | 95.8 | ND |
| PN928 | 1 μM | 3.9 | 97.3 | ND |
| | 10 μM | 2.3 | 98 | ND |
| | 100 μM | 3.2 | 98.2 | ND |
| PN926 | 1 μM | 0.9 | 97.9 | ND |
| | 10 μM | 1.4 | 98.3 | ND |
| | 100 μM | 1.2 | 98.3 | ND |
| PN931 | 1 μM | 0.6 | 97 | ND |
| | 10 μM | 0.7 | 97.7 | ND |
| | 100 μM | 0.5 | 98.4 | ND |
| PN930 | 1 μM | 0.2 | 97.9 | ND |
| | 10 μM | 1.5 | 97.2 | ND |
| | 100 μM | 0.8 | 97.8 | ND |
| PN932 | 1 μM | 0.4 | 97.3 | ND |
| | 10 μM | 1 | 96.7 | ND |
| | 100 μM | 1 | 98.4 | ND |
| PN934 | 1 μM | 0.2 | 98.3 | ND |
| | 10 μM | 1.1 | 96.2 | ND |
| | 100 μM | 0.3 | 97.4 | ND |
| PN933 | 1 μM | 0.3 | 97.7 | ND |
| | 10 μM | 0.5 | 98 | ND |
| | 100 μM | 0.2 | 98 | ND |
| PN935 | 1 μM | 20.3 | 98.1 | 1.94 |
| | 10 μM | 36.1 | 97.3 | 2.2 |
| | 100 μM | 24.6 | 96.6 | 2.16 |
| PN936 | 1 μM | 37.7 | 97.5 | 2.66 |
| | 10 μM | 55.3 | 98.1 | 5.86 |
| | 100 μM | 25.9 | 28.7 | ND |
| PN938 | 1 μM | 0.6 | 97.9 | ND |
| | 10 μM | 0.5 | 98.6 | ND |
| | 100 μM | 5.9 | 98.8 | ND |
| PN939 | 1 μM | 66 | 98.6 | 3.3 |
| | 10 μM | 97 | 99.7 | 35.1 |
| | 100 μM | 97.3 | 99.1 | 3.33 |
| PN940 | 1 μM | 69.9 | 99.1 | 4.81 |
| | 10 μM | 70.6 | 98.4 | 2.54 |
| | 100 μM | ND | ND | ND |
| PN912 | 1 μM | 61.2 | 96.9 | 4.32 |
| | 10 μM | 73.3 | 96.1 | 4.72 |
| | 100 μM | 86.2 | 90 | 5.41 |
| PN937 | 1 μM | 54.7 | 99 | 3.18 |
| | 10 μM | 92.6 | 99.4 | 6.41 |
| | 100 μM | ND | ND | ND |
| PN986 | 1 μM | 0.6 | 97.4 | ND |
| | 10 μM | 0.8 | 97.2 | ND |
| | 100 μM | 2.5 | 93.6 | ND |
| PN951 | 1 μM | 31.8 | 97.2 | 4.02 |
| | 10 μM | 80.1 | 99.1 | 14.3 |
| | 100 μM | 45.7 | 97.9 | 9.19 |
| PN969 | 1 μM | 0.6 | 98.3 | ND |
| | 10 μM | ND | ND | ND |
| | 100 μM | 15.9 | 18.6 | ND |
| PN970 | 1 μM | 93.5 | 99.3 | 29.1 |
| | 10 μM | 99.1 | 99.3 | 17.1 |
| | 100 μM | 90.3 | 91.2 | 3.77 |
| | 10 μM | 2 | 98.3 | ND |
| | 100 μM | 57.1 | 98.6 | 3.36 |
| PN947 | 1 μM | 2.8 | 98.7 | 1.23 |
| | 10 μM | 12.3 | 97.9 | 1.44 |
| | 100 μM | 49.5 | 99.3 | 4.6 |
| PN183 | 1 μM | 9.3 | 96 | 3.61 |
| | 10 μM | 47.4 | 96.8 | 3.77 |
| | 100 μM | 58.9 | 76.7 | 10.5 |

The results in Table 3 show that six of cationic peptides mediate effective intracellular delivery of siRNA without negatively impacting cell viability. As expected, the negative control siRNA alone exhibited a low percent cell-uptake and relatively low MFI indicating few cells, if any, were transfected with siRNA. In contrast, the positive control LIPOFECTAMINE™ had a percent cell-uptake of approximately 66% and a MFI of about 23, indicating that transfection of the siRNA occurred. The positive peptide control PN183 exhibited a percent cell-uptake of approximately 59% with an MFI of nearly 11. The cationic peptides that exhibited effective intracellular delivery of siRNA without negatively impacting cell viability are as follows: PN939, PN940, PN935, PN936, PN937 and PN912.

These data indicate that a cationic peptide consisting of poly-lysine or poly-lysine conjugated with a PEG moiety does not mediate effective intracellular delivery of siRNA. However, a cationic copolymer peptide with an amino acid sequence of alternating lysine and histidine residues exhibits effective intracellular delivery of siRNA. Furthermore, the addition of a PEG moiety, irrespective of size, surprisingly and unexpectedly enhances the intracellular delivery of siRNA without negatively impacting cell viability.

The cationic peptide of alternating D and L forms of arginine also exhibited effective intracellular delivery of siRNA without negatively impacting cell viability.

### Example 3

### Gene Targeted Knockdown

This Example discloses suitable methodology for determining whether cationic peptide/siRNA complexes of the present invention are capable of enhancing the ability of the siRNA to downregulate expression of one or more target genes. Example cells lines used to test peptide/siRNA complexes include 9L/LacZ and mouse tail fibroblast (MTF) cells.

SiRNA knockdown activity is determined by transfecting cells with the peptide/siRNA complexes. A random siRNA sequence may be used as a negative control.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

What is claimed is:

1. A composition for delivery of a RNA molecule to a cell, comprising:
a. a double stranded RNA (dsRNA) molecule of about 15 to about 40 base pairs; and
b. a polynucleotide delivery-enhancing peptide, comprising a region of alternating lysine and histidine residues, or of alternating D and L forms of arginine.

2. The composition of claim 1, wherein the peptide is further comprised of a polyoxyethylene group.

3. The composition of claim 2, wherein the polypeptide is polyoxyethylene glycol.

4. The composition of claim 1, wherein the nucleic acid is admixed, complexed or conjugated with the polynucleotide delivery-enhancing polypeptide.

5. The composition of claim 1, wherein said nucleic acid is a small inhibitory RNA (siRNA).

6. The composition of claim 5, wherein the nucleic acid comprises a siRNA that is complementary to a portion of a TNF-α gene or a respiratory virus nucleic acid.

7. The composition of claim 1, wherein the nucleic acid has a length of 30 or fewer nucleotides or nucleotide base pairs.

8. The composition of claim 1, wherein the polynucleotide delivery-enhancing polypeptide is selected from the group consisting of PN939, PN940, PN935, PN936, PN937 and PN912.

9. The composition of claim 8, wherein the polynucleotide delivery-enhancing polypeptide is pegylated.

10. A composition comprising a polynucleotide delivery-enhancing polypeptide and a double stranded nucleic acid, wherein said peptide comprises a region of alternating lysine and histidine residues, or of alternating D and L forms of arginine said composition causes uptake of said nucleic acid into an animal cell.

11. The composition of claim 10, wherein the nucleic acid is admixed, complexed or conjugated with the polynucleotide delivery-enhancing polypeptide.

12. The composition of claim 10, wherein said nucleic acid is a small inhibitory RNA (siRNA).

13. The composition of claim 12, wherein the nucleic acid comprises a siRNA that is complementary to a portion of a TNF-α gene.

14. The composition of claim 10, wherein the nucleic acid has a length of 30 or fewer nucleotides or nucleotide base pairs.

15. The composition of claim 10, wherein the polynucleotide delivery-enhancing polypeptide is pegylated.

16. A method for delivering a RNA molecule to a cell, comprising:
a. preparing a composition comprising:
i. a double stranded RNA (dsRNA) molecule of about 15 to about 40 base pairs;
ii. a polynucleotide delivery-enhancing peptide, comprising a region of alternating lysine and histidine residues, or of alternating D and L forms of arginine; and
b. treating a cell with said composition.

17. A method for inhibiting expression of a gene in a cell comprising:
a. preparing a pharmaceutical composition comprising:
i. a double stranded RNA (dsRNA) molecule of about 15 to about 40 base pairs, having sequence homology to a sequence of the gene;
ii. a polynucleotide delivery-enhancing peptide, comprising a region of alternating lysine and histidine residues, or of alternating D and L forms of arginine; and
b. treating a cell with said pharmaceutical composition.

18. A method for inhibiting expression of a gene in a mammal comprising:
a. preparing a pharmaceutical composition comprising:
i. a double stranded RNA (dsRNA) molecule of about 15 to about 40 base pairs, having sequence homology to a sequence of the gene;
ii. a polynucleotide delivery-enhancing peptide, comprising a region of alternating lysine and histidine residues, or of alternating D and L forms of arginine; and
b. administering said pharmaceutical composition to said mammal.

19. A method for causing uptake of a double stranded nucleic acid into an animal cell, which comprises incubating said cells with a mixture comprising a polynucleotide delivery-enhancing peptide, comprising a region of alternating lysine and histidine residues, or of alternating D and L forms of arginine and said nucleic acid.

20. The method of claim 17, wherein the nucleic acid is admixed, complexed or conjugated with the polynucleotide delivery-enhancing polypeptide.

21. The method of claim 17, wherein said nucleic acid is a small inhibitory RNA (siRNA).

22. The method of claim 21, wherein the nucleic acid comprises a siRNA that is complementary to a portion of a TNF-α gene.

23. The method of claim 17, wherein the nucleic acid has a length of 30 or fewer nucleotides or nucleotide base pairs.

24. The method of claim 1, wherein the polynucleotide delivery-enhancing polypeptide is pegylated.


SEQUENCE LISTING (14 sequences)

SEQ ID NO: 1 - RNA, 19 nt, Artificial Sequence
  Description of Artificial Sequence Synthetic oligonucleotide
ggaucuuauuucuucggag

SEQ ID NO: 2 - RNA, 19 nt, Artificial Sequence
  Description of Artificial Sequence Synthetic oligonucleotide
gggucggaacccaagcuua

SEQ ID NO: 3 - protein, 31 aa, Artificial Sequence
  Description of Artificial Sequence Synthetic peptide
WKKKKKKKKKKKKKKKKKKKKKKKKKKKKKK

SEQ ID NO: 4 - protein, 25 aa, Artificial Sequence
  Description of Artificial Sequence Synthetic peptide
KKKKKKKKKKKKKKKKKKKKKKKKK

SEQ ID NO: 5 - protein, 20 aa, Artificial Sequence
  Description of Artificial Sequence Synthetic peptide
KKKKKKKKKKKKKKKKKKKK

SEQ ID NO: 6 - protein, 15 aa, Artificial Sequence
  Description of Artificial Sequence Synthetic peptide
KKKKKKKKKKKKKKK

SEQ ID NO: 7 - protein, 10 aa, Artificial Sequence
  Description of Artificial Sequence Synthetic peptide
KKKKKKKKKK

SEQ ID NO: 8 - protein, 30 aa, Artificial Sequence
  Description of Artificial Sequence Synthetic peptide
KHKHKHKHKHKHKHKHKHKHKHKHKHKHKH

SEQ ID NO: 9 - protein, 20 aa, Artificial Sequence
  Description of Artificial Sequence Synthetic peptide
KHKHKHKHKHKHKHKHKHKH

SEQ ID NO: 10 - protein, 20 aa, Artificial Sequence
  Description of Artificial Sequence Synthetic peptide
RRRRRRRRRRRRRRRRRRRR

SEQ ID NO: 11 - protein, 31 aa, Artificial Sequence
  Description of Artificial Sequence Synthetic peptide
HHHHHHRSVCRQIKICRRRGGCYYKCTXRPY

SEQ ID NO: 12 - protein, 29 aa, Artificial Sequence
  Description of Artificial Sequence Synthetic peptide
KKKKKKKKKGLFGAIAGFIEXGWEGMIDG

SEQ ID NO: 13 - protein, 30 aa, Artificial Sequence
  Description of Artificial Sequence Synthetic peptide
KKKKKKKKSQPDIMGEWGXEIFGAIAGFLG

SEQ ID NO: 14 - protein, 28 aa, Artificial Sequence
  Description of Artificial Sequence Synthetic peptide
KETWWETWWTEWSQPGRKKRRQRRRPPQ